(19) United States
(12) Patent Application Publication
(10) Pub. No.: US 2017/0281501 A1
(43) Pub. Date: Oct. 5, 2017

(54) 4,5-DIAMINOIMIDAZOLES AS NOVEL DEVELOPER-TYPE OXIDATION DYE PRECURSORS

(71) Applicant: Henkel AG & Co. KGaA, Duesseldorf (DE)

(72) Inventors: Antje Gebert-Schwarzwalder, Neuss (DE); Helmut Giesa, Meerbusch (DE); Jan-Michael Schonebeck, Potsdam (DE)

(73) Assignee: Henkel AG & Co. KGaA, Duesseldorf (DE)

(21) Appl. No.: 15/479,136

(22) Filed: Apr. 4, 2017

(30) Foreign Application Priority Data
Apr. 5, 2016 (DE) 102016205616.6

(51) Int. Cl. A61K 8/49; C07C 211/09; C09B 57/00; C07D 233/24; C07C 211/18

(52) U.S. Cl. CPC A61K 8/4946; C07D 233/24; C07C 211/18; C09B 57/00; C07C 211/09

(57) ABSTRACT

An agent for oxidative changing of the color of keratin fibers, in particular human hair, includes, in a cosmetic support, as a developer-type oxidation dye precursor, at least one compound of formula (I)

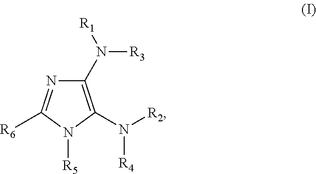

as further defined herein.

### FIELD OF THE INVENTION

The present invention generally relates to field of cosmetics and relates to agents for oxidative changing of the color of keratin-containing fibers, in particular human hairs which include 1H-imidazole-4,5-diamine and/or its derivatives as novel developer-type oxidation dye precursors.

### BACKGROUND OF THE INVENTION

Change to the form and color of hair represents an important area in modern cosmetics. Consumers use color-changing agents to achieve fashionable coloring of hair, or to conceal gray or even white hair using fashionable or natural shades of color. Depending on the requirements in terms of color or color-change, a person skilled in the art knows various systems for changing the color of human hair.

So-called oxidation coloring agents are used for permanent, intensive dyes having corresponding color fastness characteristics. Such coloring agents normally include oxidation dye precursors, so-called developer components and coupling components. The developer components form the actual dyes under the influence of oxidizing agents or atmospheric oxygen or by coupling to one or more coupling components. Oxidation coloring agents are characterized by intensive, outstanding, long-lasting coloring results. A mixture of a greater number of oxidation dye precursors can be used for naturally acting dyes, wherein in many cases, furthermore, substantive dyes are additionally used for shading.

In spite of their advantageous dyeing properties, oxidative hair coloring agents have clear disadvantages for the user. In particular, for some of the current oxidation dye precursors, including p-phenylenediamine, it is suspected of having an irritating or aggravating effect for some users, thereby triggering sensitizations or even allergic reactions. Therefore, there is still a need for further improvement of these substances in terms of physiological compatibility profile. In the search for substitute materials, many compounds have been researched which, however, suffer frequently from problems in terms of application technology, and poor color fastness characteristics. Moreover, in spite of already highly developed coloring systems, there remains a need for coloring systems which achieve brilliancy and intensity of colors, but simultaneously have a very good durability and outstanding homogeneity.

Therefore, it is desirable to reduce the aforementioned disadvantages of oxidative hair coloring agents. The coloring agents are intended to produce intensive shades with high colorfulness and with a good resistance to external influences, in particular with good fastness to light and to washing, which also do not suffer from any color reduction or color shift, even after repeated shampooing of the hair. Additionally, the dyes are intended to have an outstanding equalizing capacity and be less selective, i.e. provide the most consistent, uniform color results possible on hair pretreated in different ways. Moreover, the coloring agents are intended to possess a particularly good fastness to perspiration. Furthermore, other desirable features and characteristics of the present invention will become apparent from the subsequent detailed description of the invention and the appended claims, taken in conjunction with this background of the invention.

### BRIEF SUMMARY OF THE INVENTION

A first subject matter of the invention is an agent for oxidative changing of the color of keratin fibers, in particular human hair, including, in a cosmetic support, as a developer-type oxidation dye precursor, at least one compound of formula (I)

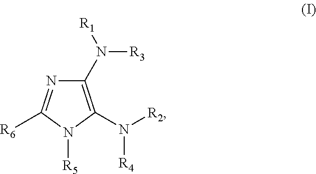

in which
R₁, R₂ independently of one another stand for hydrogen, a C₁-C₆ alkyl group, a C₂-C₆ alkenyl group, a hydroxy C₁-C₆ alkyl group, a polyhydroxy C₂-C₆ alkyl group, a C₁-C₆-alkyloxy C₂-C₆ alkyl group, an amino C₁-C₆ alkyl group, a polyamino C₂-C₆ alkyl group, a di(C₁-C₆-alkyl)amino C₁-C₆ alkyl group, a C₁-C₆ alkanoyl group, an aryl group or an aryl C₁-C₆ alkyl group,
R₃, R₄ independently of one another stand for hydrogen, a C₁-C₆ alkyl group, a C₂-C₆ alkenyl group, a hydroxy C₁-C₆ alkyl group, a polyhydroxy C₂-C₆ alkyl group, a C₁-C₆-alkyloxy C₂-C₆ alkyl group, an amino C₁-C₆ alkyl group, a polyamino C₂-C₆ alkyl group, a di(C₁-C₆-alkyl)amino C₁-C₆ alkyl group, a C₁-C₆ alkanoyl group, an aryl group or an aryl C₁-C₆ alkyl group,
R₅, R₆ independently of one another stand for hydrogen, a C₁-C₆ alkyl group, a C₂-C₆ alkenyl group, a hydroxy C1-C6 alkyl group, an aryl group or an aryl C₁-C₆ alkyl group
and/or the physiologically compatible salt thereof.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now been found that 1H-imidazole-4,5-diamine and/or its derivatives are outstandingly suited for oxidative changing of the color of keratin-containing fibers. On hair, these heterocyclic diamino compounds produce colorations with high color intensity and outstanding luster, as well as good fastness to washing and particularly good fastness to perspiration.

By keratin fibers are meant wool, felt, feathers and in particular human hair. The agents according to the invention for oxidative changing of the color can in principle also be used to color other natural fibers, such as e.g. cotton, jute, sisal, linen or silk, modified natural fibers, such as for example regenerated cellulose, nitro-, alkyl- or hydroxyalkyl- or acetyl cellulose.

The agents according to the invention are agents for oxidative changing of the color of keratin fibers, i.e. agents which are applied to keratin fibers, in particular human hairs, in order to achieve an oxidative coloring, a brightening coloring or a shading of the hair. By a brightening coloring is meant, in this context, a coloring in which the color result is brighter than the starting hair color. With a brightening coloring, in addition to at least one oxidation dye precursor of formula (I), the agents according to the invention additionally include an oxidizing agent, preferably hydrogen peroxide.

Depending on the quantities of oxidation dye precursors of formula (I) and oxidizing agent used, the oxidative changing of the color can therefore in particular be a coloring (with a high proportion of dye) or in particular a brightening (with a high proportion of oxidizing agent). In the latter case, the oxidation dye precursors are mostly used for shading the brightening result.

The agents according to the invention include compounds of formula (I) in a cosmetic support, preferably in a suitable aqueous, alcohol or aqueous-alcohol support. For the purpose of hair coloring, such supports are for example creams, emulsions, gels or also surfactant-containing foaming solutions, such as for example shampoos, foam aerosols, form formulations or other preparations which are suitable for application to hair. However, it is also conceivable to integrate the oxidation dye precursors according to formula (I) in a pulverulent or even pellet-shaped formulation.

By aqueous-alcohol solutions are meant, within the meaning of the present invention, aqueous solutions including 3 to 70 wt.-% of a C₁-C₄ alcohol, in particular ethanol or isopropanol. The agents according to the invention can additionally include further organic solvents, such as for example methoxybutanol, benzyl alcohol, ethyl diglycol or 1,2-propylene glycol. Thus all water-soluble organic solvents are preferred.

A characteristic of the agents according to the invention is the content of at least one oxidation dye precursor of formula (I). The compounds of formula (I) are developer-type oxidation dye precursors. Developer-type oxidation dye precursors (for short also called developers) are oxidized by the oxidizing agent usually present in the oxidative color-change agent, and upon this oxidation, form a reactive quinoid intermediate stage which undergoes an oxidative coupling reaction either with itself or with the couplers likewise generally included in the agent.

Oxidation coupler-type dye precursors are also called couplers for short.

Hereinafter, examples of the substituents R1, R2, R3, R4, R5 and R6 named in formula (I) are named by way of example:

Examples of C₁-C₆ alkyl residues are —CH₃, —CH₂CH₃, —CH₂CH₂CH₂CH_{3,} —CH₂CH(CH₃)₂, —CH(CH₃)CH₂CH₃, —C(CH₃)₃, —(CH₂)₄CH₃, —(CH₂)₅CH₃. Methyl and ethyl are particularly preferred alkyl residues. Examples of C₂-C₆ alkenyl groups are prop-2-enyl (allyl group), 2-methyl-prop-2-enyl, but-3-enyl, but-2-enyl, pent-4-enyl or pent-3-enyl. Examples of C₁-C₆ hydroxyalkyl groups are the hydroxymethyl group, the 2-hydroxyethyl group, the 1-hydroxyethyl group, the 3-hydroxypropyl group, the 2-hydroxypropyl group. The 2-hydroxyethyl group is particularly preferred.
Examples of C₂-C₆ polyhydroxyalkyl groups are the 2,3-dihydroxypropyl group, the 3,4-dihydroxybutyl group, the 2,4-dihydroxybutyl group and the 1,2-dihydroxyethyl group.
Examples of a C₁-C₆ alkyoxy C₁-C₆ alkyl group are the (methoxy)methyl group, the 2-(methoxy) ethyl group, the 2-(ethoxy) ethyl group, the 3-(methoxy) propyl group and the 3-(ethoxy) propyl group. The 2-(methoxy) ethyl group is particularly preferred.
Examples of an amino C₁-C₆ alkyl group are the aminomethyl group, the 2-(amino) ethyl group, the 3-(amino) propyl group and the 2-(aminopropyl) group.
Examples of a polyamino C₂-C₆ alkyl group are the 1,2-diaminoethyl group, the 2,3-diaminopropyl group, the 1,3-diaminopropyl group and the 3,4-diaminobutyl group.
Examples of a di(C₁-C₆-alkyl)amino C₁-C₆ alkyl group are the dimethylamino-methyl group, the 2-(dimethylamino) ethyl group, the 2-(diethylamino) ethyl group, the 3-(dimethylamino) propyl group and the 3-(diethylamino) propyl group. A particularly preferred example of an aryl group is the phenyl group. Examples of aryl C₁-C₆ alkyl groups are benzyl, 1-phenylethyl and 2-phenylethyl. Examples of a C₁-C₆ alkanoyl group can be called the propanoyl group (—C(O)CH₂CH₃) or the ethanoyl group (which is alternatively called the acetyl group, —C(O)CH₃). The acetyl group is a particularly preferred C₁-C₆ alkanoyl group.

With regard to the formulation of the problem according to the invention, agents which include a compound of formula (I) in which residues R1 and R2 independently of one another stand for hydrogen or a C₁-C₆ alkanoyl group show particularly advantageous effects and are therefore preferred.

In a particularly preferred embodiment, an agent according to the invention is characterized in that it includes at least one compound of formula (I) in which R1 and R2 independently of one another stand for hydrogen or a C₁-C₆ alkanoyl group.

In a particularly preferred embodiment, an agent according to the invention is characterized in that it includes at least one compound of formula (I) in which R1 and R2 independently of one another stand for hydrogen or an acetyl group (—C(O)CH₃).

Quite particularly intensive colorings can be obtained with agents which include at least one compound of formula (I) in which the residues R1 and R2 each stand for one hydrogen atom.

In a quite particularly preferred embodiment, an agent according to the invention is characterized in that it includes at least one compound of formula (I) in which R1 and R2 both stand for hydrogen.

In the course of research leading to this invention, it has been shown that the compounds of formula (I) possess quite particularly preferred properties in terms of application technology if the residues R₃ and R₄ independently of one another stand for hydrogen, a C1-C₆ alkyl group or a C₁-C₆ alkanoyl group.

In a further particularly preferred embodiment, an agent according to the invention is characterized in that it includes at least one compound of formula (I) in which R₃ and R₄ independently of one another stand for hydrogen, a C₁-C₆ alkyl group or a C₁-C₆ alkanoyl group.

In a further particularly preferred embodiment, an agent according to the invention is characterized in that it includes at least one compound of formula (I) in which R₃ and R₄ independently of one another stand for hydrogen, a C₁-C₆ alkyl group or an acetyl group (—C(O)CH₃).

Particularly preferred is also an agent for oxidative changing of the color of keratin fibers, in particular human hair, including, in a cosmetic support, as a developer-type oxidation dye precursor, at least one compound of formula (I)

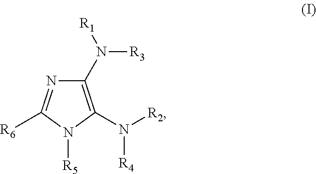

in which
R₁, R₂ independently of one another stand for hydrogen, a C₁-C₆ alkyl group or a C₁-C₆ alkanoyl group,
R₃, R₄ independently of one another stand for hydrogen, a C₁-C₆ alkyl group or a C₁-C₆ alkanoyl group,
R₅, R₆ independently of one another stand for hydrogen, a C₁-C₆ alkyl group, a hydroxy C₁-C₆ alkyl group or an aryl C₁-C₆ alkyl group, and/or the physiologically compatible salt thereof.

Particularly preferred is also an agent for oxidative changing of the color of keratin fibers, in particular human hair, including, in a cosmetic support, as a developer-type oxidation dye precursor, at least one compound of formula (I)

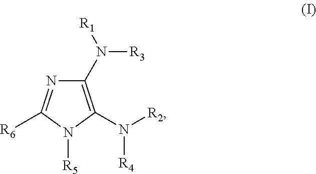

in which
R₁, R₂ independently of one another stand for hydrogen, a C₁-C₆ alkyl group or an acetyl group (—C(O)CH₃),
R₃, R₄ independently of one another stand for hydrogen, a C₁-C₆ alkyl group or an acetyl group (—C(O)CH₃),
R₅, R₆ independently of one another stand for hydrogen, a C₁-C₆ alkyl group, a hydroxy C₁-C₆ alkyl group or an aryl C₁-C₆ alkyl group,
and/or the physiologically compatible salt thereof.

Particularly preferred is also an agent for oxidative changing of the color of keratin fibers, in particular human hair, including, in a cosmetic support, as a developer-type oxidation dye precursor, at least one compound of formula (I)

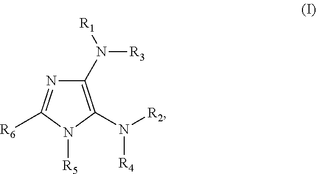

in which
R₁, R₂ independently of one another stand for hydrogen or a C₁-C₆ alkanoyl group,
R₃, R₄ independently of one another stand for hydrogen, a C₁-C₆ alkyl group or a C₁-C₆ alkanoyl group,
R₅, R₆ independently of one another stand for hydrogen, a C₁-C₆ alkyl group, a hydroxy C₁-C₆ alkyl group or an aryl C₁-C₆ alkyl group, and/or the physiologically compatible salt thereof.

Particularly preferred is also an agent for oxidative changing of the color of keratin fibers, in particular human hair, including, in a cosmetic support, as a developer-type oxidation dye precursor, at least one compound of formula (I)

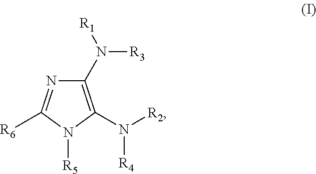

in which
R₁, R₂ independently of one another stand for hydrogen or an acetyl group (—C(O)CH₃),
R₃, R₄ independently of one another stand for hydrogen, a C₁-C₆ alkyl group or an acetyl group (—C(O)CH₃),
R₅, R₆ independently of one another stand for hydrogen, a C₁-C₆ alkyl group, a hydroxy C₁-C₆ alkyl group or an aryl C₁-C₆ alkyl group, and/or the physiologically compatible salt thereof.

Furthermore, compounds of formula (I) are preferred in which R₅ stands for hydrogen or for a C₁-C₆ alkyl group.

In a further particularly preferred embodiment, an agent according to the invention is characterized in that it includes at least one compound of formula (I) in which R₅ stands for hydrogen or for a C₁-C₆ alkyl group.

Compounds of formula (I) in which R₅ stands for a methyl group or an ethyl group are quite particularly preferred.

In a further particularly preferred embodiment, an agent according to the invention is characterized in that it includes at least one compound of formula (I) in which R₅ stands for a methyl group or an ethyl group.

Particularly preferred is also an agent for oxidative changing of the color of keratin fibers, in particular human hair, including, in a cosmetic support, as a developer-type oxidation dye precursor, at least one compound of formula (I)

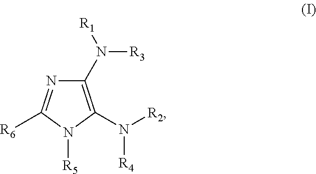

in which
R₁, R₂ independently of one another stand for hydrogen or a C₁-C₆ alkanoyl group,
R₃, R₄ independently of one another stand for hydrogen, a C₁-C₆ alkyl group or a C₁-C₆ alkanoyl group,
R₅, R₆ independently of one another stand for hydrogen or a C₁-C₆ alkyl group, and/or the physiologically compatible salt thereof.

Particularly preferred is also an agent for oxidative changing of the color of keratin fibers, in particular human hair, including, in a cosmetic support, as a developer-type oxidation dye precursor, at least one compound of formula (I)

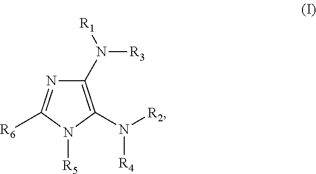

in which
R₁, R₂ independently of one another stand for hydrogen or a C₁-C₆ alkanoyl group,
R₃, R₄ independently of one another stand for hydrogen, a methyl group, an ethyl group or a C₁-C₆ alkanoyl group,
R₅, R₆ independently of one another stand for hydrogen, a methyl group or an ethyl group,
and/or the physiologically compatible salt thereof.

Particularly preferred is also an agent for oxidative changing of the color of keratin fibers, in particular human hair, including, in a cosmetic support, as a developer-type oxidation dye precursor, at least one compound of formula (I)

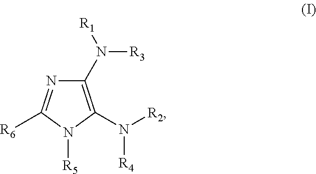

in which
R₁, R₂ independently of one another stand for hydrogen or an acetyl group (—C(O)CH₃),
R₃, R₄ independently of one another stand for hydrogen, a methyl group, an ethyl group or an acetyl group (—C(O)CH₃),
R₅, R₆ independently of one another stand for hydrogen, a methyl group or an ethyl group,
and/or the physiologically compatible salt thereof.

Particularly intensive dyes are obtained if the residue R₆ stands for hydrogen in the compounds of formula (I).

In a further particularly preferred embodiment, an agent according to the invention is characterized in that it includes at least one compound of formula (I) in which R₆ stands for hydrogen.

Particularly preferred is also an agent for oxidative changing of the color of keratin fibers, in particular human hair, including, in a cosmetic support, as a developer-type oxidation dye precursor, at least one compound of formula (I)

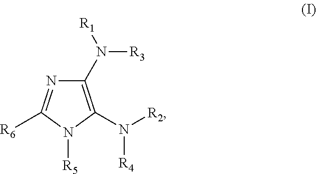

in which
R₁, R₂ independently of one another stand for hydrogen or a C1-C₆ alkanoyl group,
R₃, R₄ independently of one another stand for hydrogen, a methyl group, an ethyl group or a C₁-C₆ alkanoyl group,
R₅ stands for hydrogen, a methyl group or an ethyl group,
R₆ stands for hydrogen,
and/or the physiologically compatible salt thereof.

Particularly preferred is also an agent for oxidative changing of the color of keratin fibers, in particular human hair, including, in a cosmetic support, as a developer-type oxidation dye precursor, at least one compound of formula (I)

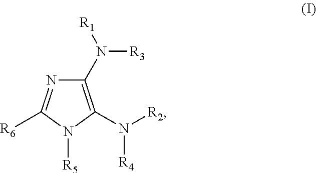

in which
R₁, R₂ independently of one another stand for hydrogen or an acetyl group (—C(O)CH₃),
R₃, R₄ independently of one another stand for hydrogen, a methyl group, an ethyl group or an acetyl group (—C(O)CH₃),
R₅ stands for hydrogen, a methyl group or an ethyl group,
R₆ stands for hydrogen,
and/or the physiologically compatible salt thereof.

In a further quite particularly preferred embodiment, an agent according to the invention is characterized in that it includes at least one compound of formula (I) which is selected from the group of
1H-imidazole-4,5-diamine

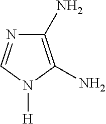

1-methyl-1H-imidazole-4,5-diamine

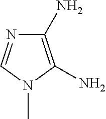

1-ethyl-1H-imidazole-4,5-diamine

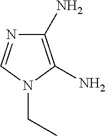

4-N-methyl-1H-imidazole-4,5-diamine

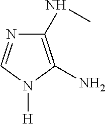

1,4-N-dimethyl-1H-imidazole-4,5-diamine

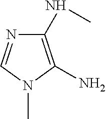

1-ethyl-4-N-methyl-1H-imidazole-4,5-diamine

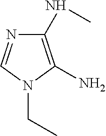

4-N-ethyl-1H-imidazole-4,5-diamine

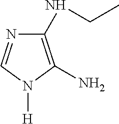

4-N-methyl-1H-imidazole-4,5-diamine

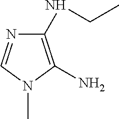

1,4-N-diethyl-1H-imidazole-4,5-diamine

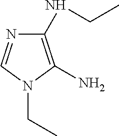

5-N-methyl-1H-imidazole-4,5-diamine

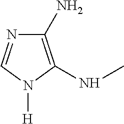

1,5-N-dimethyl-1H-imidazole-4,5-diamine

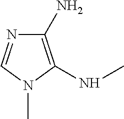

1-ethyl-5-N-methyl-1H-imidazole-4,5-diamine

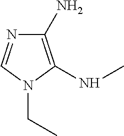

5-N-ethyl-1-N-imidazole-4,5-diamine

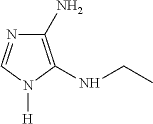

5-N-ethyl-1-methyl-1H-imidazole-4,5-diamine

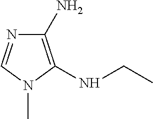

1,5-N-diethyl-1H-imidazole-4,5-diamine

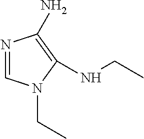

4-N,5-N-dimethyl-1H-imidazole-4,5-diamine

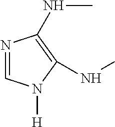

1,4-N,5-N-trimethyl-1H-imidazole-4,5-diamine

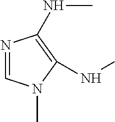

1-ethyl-4-N,5-N-dimethyl-1H-imidazole-4,5-diamine

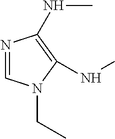

4-N-ethyl-5-N-methyl-1H-imidazole-4,5-diamine

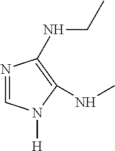

4-N-ethyl-1,5-N-dimethyl-1H-imidazole-4,5-diamine

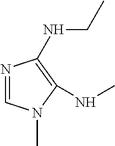

1,4-N-dimethyl-5-N-methyl-1H-imidazole-4,5-diamine

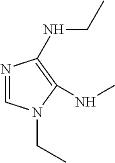

5-N-ethyl-4-N-methyl-1H-imidazole-4,5-diamine

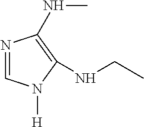

5-N-ethyl-1,4-N-dimethyl-1H-imidazole-4,5-diamine

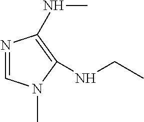

1,5-N-diethyl-4-N-methyl-1H-imizadole-4,5-diamine

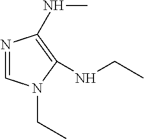

4-N,5-N-diethyl-1H-imidazole-4,5-diamine

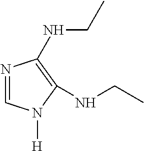

4-N,5-N-diethyl-1-methyl-1H-imidazole-4,5-diamine

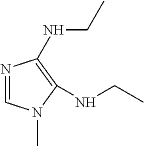

1,4-N,5-N-triethyl-1H-imidazole-4,5-diamine

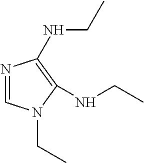

N-(5-amino-1H-imidazole-4-yl)acetamide

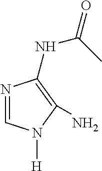

N-(5-amino-1-methyl-1H-imidazole-4-yl)acetamide

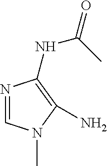

N-(5-amino-1-ethyl-1H-imidazole-4-yl)acetamide

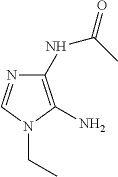

N-(4-amino-1H-imidazole-5-yl)acetamide

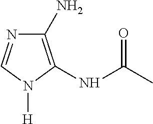

N-(4-amino-1-methyl-1H-imidazole-5-yl)acetamide

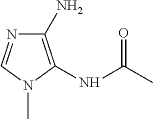

N-(4-amino-1-ethyl-1H-imidazole-5-yl)acetamide

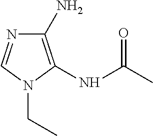

N-acetyl-N-(5-amino-1H-imidazole-4-yl)acetamide

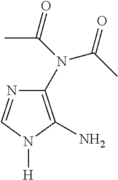

N-acetyl-N-(5-amino-1-methyl-1H-imidazole-4-yl)acetamide

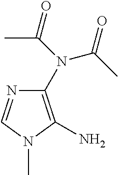

N-acetyl-N-(5-amino-1-ethyl-1H-imidazole-4-yl)acetamide

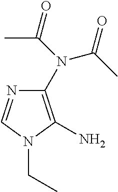

N-acetyl-N-(4-amino-1H-imidazole-5-yl)acetamide

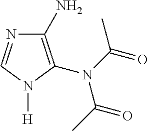

N-acetyl-N-(4-amino-1-methyl-1H-imidazole-5-yl)acetamide

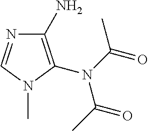

N-acetyl-N-(4-amino-1-ethyl-1H-imidazole-5-yl)acetamide

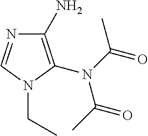

and/or the physiologically compatible salts of these compounds.

In a further explicitly quite particularly preferred embodiment, an agent according to the invention is characterized in that it includes at least one compound of formula (I) which is selected from the group of

1H-imidazole-4,5-diamine

1-methyl-1H-imidazole-4,5-diamine

1-ethyl-1H-imidazole-4,5-diamine

N-(5-amino-1H-imidazole-4-yl) acetamide

N-(5-amino-1-methyl-1H-imidazole-4-yl) acetamide

N-(5-amino-1-ethyl-1H-imidazole-4-yl) acetamide

N-(4-amino-1H-imidazole-5-yl) acetamide

N-(4-amino-1-methyl-1H-imidazole-5-yl) acetamide

N-(4-amino-1-ethyl-1H-imidazole-5-yl) acetamide

and/or the physiologically compatible salts of these compounds.

The compounds according to the invention of formula (I) are amino compounds. These can also be used in the form of the physiologically compatible salts thereof, in particular chlorides, sulfates and bromides. Further preferred salts are derived from sulfonic acids, such as benzenesulfonates, p-toluene sulfonates, C₁-C₄ alkane sulfonates or trifluoromethane sulfonates. Depending on the number of amino groups included in the compounds according to the invention, mono-, di-, tri-, tetra- and higher adducts may be present as salts.

By the physiologically compatible salts of compounds of formula (I) are quite particularly preferably meant the chloride salts, sulfate salts and bromide salts of these compounds.

Agents preferred according to the invention are characterized in that they include one or more compounds of formula (I) in a total quantity of from 0.001 to 5.0 wt.-%, preferably of from 0.025 to 2.5 wt.-%, particularly preferably of from 0.05 to 2.0 wt.-% and in particular preferably of from 0.1 to 1.5 wt. %, relative to the total weight of the agent.

In a further preferred embodiment, the agent according to the invention is characterized in that it includes one or more compounds of formula (I) in a total quantity of from 0.001 to 5.0 wt.-%, preferably of from 0.025 to 2.5 wt.-%, particularly preferably of from 0.05 to 2.0 wt.-% and in particular preferably of from 0.1 to 1.5 wt.-%, relative to the total weight of the agent.

The compounds of formula (I) can be included in the agent according to the invention as compounds which change color by their own action. As the compounds of formula (I) are developer-type oxidation dye precursors, in this case the dyes form due to self-reaction. However, according to the invention it is preferred if the agent additionally includes at least one coupler-type oxidation dye precursor (called “coupler” for short).

Within the framework of the oxidative coloring, coupling components on their own do not form any significant dye, but always require the presence of developer components. Within the framework of the invention, coupling components permit at least one substitution of a chemical residue of the coupler due to the oxidized form of the developer component. In so doing, covalent bonds form between coupler and developer component.

Preferably at least one compound from the following classes is selected as a coupling component which is suitable according to the invention:

m-aminophenol and/or its derivatives,

m-diaminobenzene and/or its derivatives,

o-diaminobenzene and/or its derivatives,

o-aminophenol derivatives, such as for example o-aminophenol,

naphthalene derivatives with at least one hydroxy group,

di- or trihydroxybenzene and/or derivatives thereof,

pyridine derivatives,

pyrimidine derivatives,

monohydroxyindole derivatives and/or monoaminoindole derivatives,

monohydroxyindole derivatives and/or monoaminoindole derivatives,

pyrazolone derivatives, such as for example 1-phenyl-3-methyl pyrazol-5-one,

morpholine derivatives, such as for example 6-hydroxybenzomorpholine or 6-aminobenzomorpholine,

quinoxaline derivatives, such as for example 6-methyl-1,2,3,4-tetrahydroquinoxaline.

Mixtures of two or more compounds from one or more of these classes are likewise according to the invention within the framework of this embodiment.

In a further preferred embodiment, an agent according to the invention is characterized in that it additionally includes at least one coupler-type oxidation dye precursor, which is selected from the group of 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)-amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[[(2-hydroxyethyl)amino]-4,5-dimethylphenyl}amino)ethanol, 2-[3-morpholine-4-ylphenyl)amino]ethanol, 3-amino-4-(2-methoxyethoxy)-5-methylphenylamine, 1-amino-3-bis-(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methyl pyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindolin, 6-hydroxyindolin, 7-hydroxyindolin and the physiologically compatible salts thereof.

Quite particularly preferred are resorcinol, 2-methylresorcinol, 5-amino-2-methylphenol, 3-aminophenol, 2-(2,4-diaminophenoxy)ethanol, 1,3-bis(2,4-diaminophenoxy)propane, 1-methoxy-2-amino-4-(2′-hydroxyethylamino)benzene, 2-amino-3-hydroxypyridine and/or 1-naphthol and/or the physiologically compatible salts thereof.

The coupling components are preferably used in a quantity of from 0.0001 to 10 wt.-%, preferably 0.01 to 5.0 wt.-%, in each case relative to the total weight of the agent according to the invention.

Particularly preferred are agents for oxidative discoloration of keratin fibers, including 1H-imidazole-4,5-diamine and at least one compound selected from the group of 5-amino-2-methylphenol, 3-amino-2-chloro-6-methylphenol, 2,4-dichloro-3-aminophenol, 2-(2,4-diaminophenoxy)ethanol, 1,3 -bis(2,4-diaminophenoxy)propane, resorcinol, 2-methylresorcinol, 2-amino-3-hydroxypyridine, 3-amino-2-methylamino-6-methoxypyridine, 1-naphthol, 1,5-dihydroxynaphthalene, 2,7-dihydroxynaphthalene or
1-methyl-1H-imidazole-4,5-diamine and at least one compound selected from the group of 5-amino-2-methylphenol, 3-amino-2-chloro-6-methylphenol, 2,4-dichloro-3-aminophenol, 2-(2,4-diaminophenoxy)ethanol, 1,3-bis(2,4-diaminophenoxy)propane, resorcinol, 2-methylresorcinol, 2-amino-3-hydroxypyridine, 3-amino-2-methylamino-6-methoxypyridine, 1-naphthol, 1,5-dihydroxynaphthalene, 2,7-dihydroxynaphthalene or
1-ethyl-1H-imidazole-4,5-diamine and at least one compound selected from the group of 5-amino-2-methylphenol, 3-amino-2-chloro-6-methylphenol, 2,4-dichloro-3-aminophenol, 2-(2,4-diaminophenoxy)ethanol, 1,3-bis(2,4-diaminophenoxy)propane, resorcinol, 2-methylresorcinol, 2-amino-3-hydroxypyridine, 3-amino-2-methylamino-6-methoxypyridine, 1-naphthol, 1,5-dihydroxynaphthalene, 2,7-dihydroxynaphthalene or
N-(5-amino-1H-imidazole-4-yl) acetamide and at least one compound selected from the group of 5-amino-2-methylphenol, 3-amino-2-chloro-6-methylphenol, 2,4-dichloro-3-aminophenol, 2-(2,4-diaminophenoxy)ethanol, 1,3-bis(2,4-diaminophenoxy)propane, resorcinol, 2-methylresorcinol, 2-amino-3-hydroxypyridine, 3-amino-2-methylamino-6-methoxypyridine, 1-naphthol, 1,5-dihydroxynaphthalene, 2,7-dihydroxynaphthalene or
N-(5-amino-1-methyl-1H-imidazole-4-yl) acetamide and at least one compound selected from the group of 5-amino-2-methylphenol, 3-amino-2-chloro-6-methylphenol, 2,4-dichloro-3-aminophenol, 2-(2,4-diaminophenoxy)ethanol, 1,3-bis(2,4-diaminophenoxy)propane, resorcinol, 2-methylresorcinol, 2-amino-3-hydroxypyridine, 3-amino-2-methylamino-6-methoxypyridine, 1-naphthol, 1,5-dihydroxynaphthalene, 2,7-dihydroxynaphthalene or
N-(5-amino-1-ethyl-1H-imidazole-4-yl) acetamide and at least one compound selected from the group of 5-amino-2-methylphenol, 3-amino-2-chloro-6-methylphenol, 2,4-dichloro-3-aminophenol, 2-(2,4-diaminophenoxy)ethanol, 1,3-bis(2,4-diaminophenoxy)propane, resorcinol, 2-methylresorcinol, 2-amino-3-hydroxypyridine, 3-amino-2-methylamino-6-methoxypyridine, 1-naphthol, 1,5-dihydroxynaphthalene, 2,7-dihydroxynaphthalene or
N-(4-amino-1H-imidazole-5-yl) acetamide and at least one compound selected from the group of 5-amino-2-methylphenol, 3-amino-2-chloro-6-methylphenol, 2,4-dichloro-3-aminophenol, 2-(2,4-diaminophenoxy)ethanol, 1,3-bis(2,4-diaminophenoxy)propane, resorcinol, 2-methylresorcinol, 2-amino-3-hydroxypyridine, 3-amino-2-methylamino-6-methoxypyridine, 1-naphthol, 1,5-dihydroxynaphthalene, 2,7-dihydroxynaphthalene or
N-(4-amino-1-methyl-1H-imidazole-5-yl) acetamide and at least one compound selected from the group of 5-amino-2-methylphenol, 3-amino-2-chloro-6-methylphenol, 2,4-dichloro-3-aminophenol, 2-(2,4-diaminophenoxy)ethanol, 1,3-bis(2,4-diaminophenoxy)propane, resorcinol, 2-methylresorcinol, 2-amino-3-hydroxypyridine, 3-amino-2-methylamino-6-methoxypyridine, 1-naphthol, 1,5-dihydroxynaphthalene, 2,7-dihydroxynaphthalene or
N-(4-amino-1-ethyl-1H-imidazole-5-yl) acetamide and at least one compound selected from the group of 5-amino-2-methylphenol, 3-amino-2-chloro-6-methylphenol, 2,4-dichloro-3-aminophenol, 2-(2,4-diaminophenoxy)ethanol, 1,3-bis(2,4-diaminophenoxy)propane, resorcinol, 2-methylresorcinol, 2-amino-3-hydroxypyridine, 3-amino-2-methylamino-6-methoxypyridine, 1-naphthol, 1,5-dihydroxynaphthalene, 2,7-dihydroxynaphthalene.

In order to achieve a balanced and subtle formation of hue, it is advantageous, according to the invention, if the agent according to the invention includes further chromophoric components.

Therefore, it may be preferred, according to the invention, if the agent includes at least one further chromophoric component which is selected from additional developer-type oxidation dye precursors and/or substantive dyes.

In addition to the developer-type oxidation dye precursors according to formula (I), the agents according to the invention can therefore additionally still include at least one further developer component.

Preferred further developer components are selected from the group of p-phenylenediamine, p-toluenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3-(1H-imidazole-1-yl)propyl]amine, N,N′-bis-(2-hydroxyethyl)-N,N′-bis-(4-aminophenyl)-1,3-diamino-propane-2-ol, Bis-(2-hydroxy-5-aminophenyl)methane, 1,3-bis-(2,5-diaminophenoxy)-propane-2-ol, N,N′-bis-(4-aminophenyl)-1,4-diazacycloheptane, 1,10-bis-(2,5-diaminophenyl)-1,4,7,10-tetraoxadecane, 4-aminophenol, 4-amino-3-methylphenol, 4-amino-2-aminomethylphenol, 4-amino-2-(1,2-dihydroxyethyl)phenol and 4-amino-2-(diethylaminomethyl)phenol, 4,5-diamino-1-(2-hydroxyethyl)pyrazol, 2,4,5,6-tetraaminopyrimidine, 4-hydroxy-2,5,6-triaminopyrimidine, 2-hydroxy-4,5,6-triaminopyrimidine, as well as the physiologically compatible salts of these compounds. Particularly preferred additional developer components are p-toluenediamine, 2-(2-hydroxyethyl)-p-phenylenediamine, 2-methoxymethyl-p-phenylenediamine, N-(4-amino-3-methylphenyl)-N-[3-(1H-imidazole-1-yl)propyl]amine and/or 4,5-diamino-1-(2-hydroxyethyl)-pyrazol as well as the physiologically compatible salts thereof.

In a further preferred embodiment an agent according to the invention is characterized in that it additionally includes at least one developer-type oxidation dye precursor which is selected from the group of p-phenylenediamine, p-toluenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3-(1H-imidazole-1-yl)propyl]amine, N,N′-bis-(2-hydroxyethyl)-N,N′-bis-(4-aminophenyl)-1,3-diamino-propane-2-ol, bis-(2-hydroxy-5-aminophenyl)methane, 1,3-bis-(2,5-diaminophenoxy)-propane-2-ol, N,N′-bis-(4-aminophenyl)-1,4-diazacycloheptane, 1,10-bis-(2,5-diaminophenyl)-1,4,7,10-tetraoxadecane, 4-aminophenol, 4-amino-3-methylphenol, 4-amino-2-aminomethylphenol, 4-amino-2-(1,2-dihydroxyethyl)phenol and 4-amino-2-(diethylaminomethyl)phenol, 4,5-diamino-1-(2-hydroxyethyl)pyrazol, 2,4,5,6-tetraaminopyrimidine, 4-hydroxy-2,5,6-triaminopyrimidine, 2-hydroxy-4,5,6-triaminopyrimidine, as well as the physiologically compatible salts of these compounds. Particularly preferred additional developer components are p-toluenediamine, 2-(2-hydroxyethyl)-p-phenylenediamine, 2-methoxymethyl-p-phenylenediamine, N-(4-amino-3-methylphenyl)-N-[3-(1H-imidazole-1-yl)propyl]amine, 4,5-diamino-1-(2-hydroxyethyl)-pyrazol and the physiologically compatible salts thereof.

The additional developer components are preferably used in a quantity of from 0.0001 to 10 wt.-%, preferably 0.001 to 5 wt.-%, in each case relative to the total weight of the agent according to the invention.

Furthermore, the agents according to the invention can include at least one substantive dye. These are dyes which adhere directly to the hair and do not require any oxidative process for forming the color. Substantive dyes are usually nitrophenylenediamine, nitroaminophenole, azo dyes, anthraquinones, triarylmethane dyes or indophenol.

The substantive dyes are each preferably used in a quantity of from 0.001 to 20 wt.-%, in particular of from 0.05 to 5 wt.-%, in each case relative to the total weight of the agent according to the invention. The total quantity of substantive dyes is preferably at most 3 wt.-%.

Substantive dyes can be divided into anionic, cationic and non-ionic substantive dyes, which are selected and used by a person skilled in the art according to the requirements of the support base.

Preferred anionic substantive dyes are those compounds known under the international denominations or trade names Bromophenol blue, Tetrabromophenol blue, Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1 and Acid Black 52.

Preferred cationic substantive dyes are Basic Blue 7, Basic Blue 26, Basic Violet 2 and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Yellow 87, Basic Orange 31 and Basic Red 51.

In particular, non-ionic nitro dyes and quinone dyes and neutral azo dyes are suitable as non-ionic substantive dyes. Preferred non-ionic substantive dyes are the compounds known under the international denominations or trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)amino-4-nitrobenzene, 2-[(4-amino-2-nitrophenyl)amino]-benzoic acid, 6-nitro-1,2,3,4-tetrahydroquinoxaline, 2-hydroxy-1,4-naphthoquinone, picramic acid and the salts thereof, 2-amino-6-chloro-4-nitrophenol, 4-ethylamino-3-nitrobenzoic acid and 2-chloro-6-ethylamino-4-nitrophenol.

In addition to the compound according to formula (I), the agents according to the invention can also include natural analog dyes. Compounds according to the invention which include precursors of natural analog dyes are preferably used as air-oxidative coloring agent. In this embodiment, an additional oxidizing agent is not added to said compositions.

Particularly well suited as precursors of natural analog hair dyes are derivatives of 5,6-dihydroxyindoles, in particular 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole as well as 5,6-dihydroxyindole-2-carboxylic acid, as well as furthermore derivatives of 5,6-dihydroxyindole, in particular 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, 5,6-dihydroxyindole-2-carboxylic acid, as well as physiologically compatible salts of the aforementioned compounds.

The dye precursors of natural analog dyes are each used preferably in a quantity of from 0.001 to 5 wt.-%, relative to the total weight of the agent according to the invention.

In the case of oxidative colorings, the color can in principle develop with atmospheric oxygen. Preferably, however, a chemical oxidizing agent is used, particularly if, in addition to the dye, a brightening effect is desired on human hair. This brightening effect can be desired, regardless of the coloring method. As oxidizing agents there come into question persulfates, peroxodisulfates, chlorites, hypochlorites and in particular hydrogen peroxide and/or one of its solid products of addition to organic or inorganic compounds. The use of hydrogen peroxide is quite particularly preferred as oxidizing agent.

In a further quite particularly preferred, an agent according to the invention is characterized in that it additionally includes hydrogen peroxide.

In order to prevent a premature, undesired reaction of the oxidation dye precursors by the oxidizing agent, oxidation dye precursors and oxidizing agent itself are expediently manufactured separately and then brought into contact with one another directly before application.

In a further embodiment of the present invention, therefore, agents are preferred which are characterized in that they are produced directly before application, by mixing at least two preparations, wherein the at least two preparations are prepared, manufactured in at least two separate containers, and wherein a container includes a coloring agent (A) which includes at least one compound of formula (I) in a cosmetic support as a developer-type oxidation dye precursor, and a further container includes an oxidizing agent preparation (B), including at least one oxidizing agent.

Preferably, the oxidizing agent preparation (B) includes hydrogen peroxide and/or one of its solid products of addition to organic or inorganic compounds, such as urea, melamine as well as sodium borate as oxidizing agent.

Preferably the quantity of oxidizing agent in the agent according to the invention is 0.5 to 12 wt.-%, preferably 2 to 10 wt.-%, in particular preferably 3 to 6 wt.-% (calculated as 100% H₂O₂). If the agent according to the invention includes hydrogen peroxide, then the agent is the application-ready agent which was obtained by mixing the coloring agent (A) which includes at least one oxidation dye precursor according to formula (I) in a cosmetic support with an oxidizing agent preparation (B) which includes hydrogen peroxide.

Such oxidizing agent preparations are preferably aqueous, pourable oxidizing agent preparations. In doing so, preferred preparations are characterized in that the pourable oxidizing agent preparation includes 40 to 90 wt.-%, preferably 50 to 85 wt.-%, particularly preferably 55 to 80 wt.-%, further preferably 60 to 77.5 wt.-% and in particular 65 to 75 wt.-% water, relative to its weight.

Furthermore, it has proved advantageous if the oxidizing agent preparations include at least one stabilizer or complexer. Complexers and stabilizers which are conventional and preferred within the framework of the present invention are for example polyoxycarboxylic acids, polyamines, ethylenediaminetetraacetic acid (EDTA), N-hydroxyethylethylenediaminetriacetic acid, diethylenetriaminpentaacetic acid (DTPA), ethylenediaminedisuccinic acid (EDDS), hydroxyethyliminodiacetic acid, nitridodiacetic acid-3-propionic acid, isoserine diacetic acid, N,N-di-(2-hydroxyethyl)glycine, N-(1,2-dicarboxy-2-hydroxyethyl)glycine, N-(1,2-dicarboxy-2-hydroxyethyl)aspartic acid or nitrilotriacetic acid (NTA), ethylenediamine diglutaric acid (EDGA), 2-hydroxypropylenediamine disuccinic acid (HPDS), glycinamide-N,N′-disuccinic acid (GADS), ethylenediamine-N-N′-diglutaric acid (EDDG), 2-hydroxypropylenediamine-N-N′-disuccinic acid (HPDDS), diaminoalkyldi-(sulfosuccinic acid) (DDS), ethylene dicysteic acid (EDC), ethylenediamine-N-N′-bis(ortho-hydroxyphenyl) acetic acid (EDDHA), N-2-hydroxyethylamine-N,N-diacetic acid, glyceryl iminodiacetic acid, iminodiacetic acid-N-2-hydroxypropyl sulfonic acid, aspartic acid-N-carboxymethyl-N-2,5-hydroxypropyl-3-sulfonic acid, β-alanine-N,N′-diacetic acid, aspartic acid-N,N′-diacetic acid, aspartic acid-N-monoacetic acid, dipicolic acid, as well as their salts and/or derivatives, geminal diphosphonic acids such as 1-hydroxyethane-1,1-diphosphonic acid (HEDP), the higher homologs thereof with up to 8 carbon atoms as well as hydroxy or amino-group-containing derivatives hereof and 1-aminoethane-1,1-diphosphonic acid, the higher homologs thereof with up to 8 carbon atoms as well as hydroxy or amino-group-containing derivatives, aminophosphonic acids such as ethylenediaminetetra(methylenephosphonic acid) (EDTMP), diethylene-triaminepenta(methylenephosphonic acid) (DTPMP) as well as the higher homologs thereof, or nitrilotri(methylenephosphonic acid), phosphonopolycarboxylic acids such as 2-phosphonobutane-1,2,4-tricarboxylic acid, cyclodextrines, such as alkali stannate (sodium stannate), alkalipyrophosphates (tetrasodium pyrophosphate, disodium pyrophosphate), alkaliphosphates (sodium phosphate), and phosphoric acid as well as the salts thereof.

In a further quite particularly preferred embodiment, an agent according to the invention is characterized in that it includes at least one compound, which is selected from the group of ethylenediaminetetraacetic acid (EDTA), N-hydroxyethylethylenediaminetriacetic acid, diethylenetriaminpentaacetic acid (DTPA), ethylenediaminedisuccinic acid (EDDS), N,N-di-(2-hydroxyethyl)glycine, ethylenediaminediglutaric acid (EDGA), 2-hydroxypropylendiaminedisuccinic acid (HPDS), glycinamide-N,N′-disuccinic acid (GADS), ethylenediamine-N-N′-diglutaric acid (EDDG), 2-hydroxypropylenediamine-N-N′-disuccinic acid (HPDDS), diaminoalkyldi-(sulfosuccinic acid) (DDS), ethylenedicysteic acid (EDC), ethylenediamine-N-N′-bis(ortho-hydroxyphenyl) acetic acid (EDDHA), dipicolinic acid, 1-hydroxyethane-1,1-diphosphonic acid (HEDP), ethylenediaminetetra(methylenephosphonic acid) (EDTMP), diethylene-triaminepenta(methylenephosphonic acid) (DTPMP) and/or the physiologically compatible salts thereof.

In the alkali pH values required according to the invention of the treatment solutions, these complexers are present at least partially as anions. It does not matter whether they are introduced in the form of acids or in the form of salts. In the case of use as salts, alkali-, ammonium or alkylammonium salts, in particular sodium salts, are preferred.

Complexers preferred according to the invention are nitrogen-containing polycarboxylic acids, in particular EDTA, and phosphonates, preferably hydroxyalkane or aminoalkane phosphonates and in particular 1-hydroxyethane-1,1-diphosphonate (HEDP) or the di- or tetra sodium salt thereof, and/or ethylenediaminetetramethylenephosphonate (EDTMP) or the hexasodium salt thereof, and/or diethylenetriaminepentamethylenephosphonate (DTPMP) or the hepta- or octasodium salt thereof.

Oxidative dyeing processes on keratin fibers usually occur in the alkali medium. In order to preserve the keratin fibers and also the skin as far as possible, it is, however, not desirable to set too high a pH. Therefore it is preferred if the pH of the application-ready agent is in the range of from 7.5 to 12.5, preferably of from 8.0 to 12.0, further preferably of from 8.5 to 11.5 and quite particularly preferably of from 9,0 to 10.5. The pH values within the meaning of the present invention are pH values which have been measured at a temperature of 22° C.

In a quite particularly preferred embodiment, an agent according to the invention is characterized in that it includes water and has a pH in the range of from 7.5 to 12.5, preferably of from 8.0 to 12.0, further preferably of from 8.5 to 11.5 and quite particularly preferably of from 9.0 to 10.5.

Normally, the pH is set using pH-setting agents. Acidification and alkalization agents are familiar to a person skilled in the art of cosmetics for setting the pH. The alkalization agents which can be used to set pH are typically chosen from inorganic salts, organic alkalization agents, in particular amines, basic amino acids and alkanolamines, and ammonia. Acidification agents preferred according to the invention are culinary acids, for example citric acid, acetic acid, malic acid or tartaric acid, as well as diluted mineral acids. The pH values within the meaning of the present invention are pH values which have been measured at a temperature of 22° C.

Organic alkalization agents which can be used according to the invention are preferably selected from alkanolamines from primary, secondary or tertiary amines with a C₂-C₆ alkyl base which has at least one hydroxyl group. Particularly preferred alkanolamines are selected from the group 2-aminoethane-1-ol (monoethanolamine), 2-amino-2-methylpropane-1-ol, 2-amino-2-methyl-propane-1,3-diol and triethanolamine.

Within the framework of the experiments for the present invention, it transpires that further agents suitable according to the invention are characterized in that they additionally include an inorganic alkalization agent. The inorganic alkalization agent according to the invention is preferably selected from the group which is formed from sodium hydroxide, potassium hydroxide, calcium hydroxide, barium hydroxide, sodium phosphate, potassium phosphate, sodium silicate, potassium silicate, sodium carbonate and potassium carbonate.

A further quite particularly preferred alkalization agent is ammonia.

In a quite particularly preferred embodiment, an agent according to the invention is characterized in that it includes at least one alkalization agent from the group of ammonia, 2-aminoethane-1-ol (monoethanolamine), 2-amino-2-methylpropane-1-ol, 2-amino-2-methyl-propane-1,3-diol, triethanolamine, arginine, histidine and/or lysine.

Preferably, the alkalization agents are used in a quantity of from 0.05 to 10 wt.-%, in particular of from 0.5 to 5 wt.-%, in each case relative to the total weight of the agent according to the invention.

Furthermore, the agents according to the invention can include further active ingredients, auxiliaries and additives such as for example non-ionic polymers such as for example vinylpyrrolidinone/vinylacrylate copolymers, polyvinylpyrrolidinone, vinylpyrrolidinone/vinylacetate copolymers, polyethylene glycols and polysiloxanes; additional silicones such as volatile or non-volatile, linear, branched or cyclical, crosslinked or non-crosslinked polyalkylsiloxanes (such as dimethicones or cyclomethicones), polyarylsiloxanes and/or polyalkylarylsiloxanes, in particular polysiloxanes with organofunctional groups, such as substituted or unsubstituted amines (amodimethicones), carboxyl, alkoxy and/or hydroxyl groups (dimethicone copolyols), linear polysiloxane(A)-polyoxyalkylene(B)-block copolymers, grafted silicone polymers; cationic polymers such as quaternized cellulose ethers, polysiloxanes with quaternary groups, dimethyldiallylammonium chloride polymers, acrylamide-dimethyldiallyl-ammonium chloride copolymers, with diethylsulfate quaternized dimethylamino-ethyl methacrylate-vinylpyrrolidinone copolymers, vinylpyrrolidinone-imidazolinium-methochloride copolymers and quaternized polyvinylalcohol; zwitterionic and amphoteric polymers; anionic polymers such as for example polyacrylic acids or crosslinked polyacrylic acids; anionic surfactants, zwitterionic surfactants, amphoteric surfactants, non-ionic surfactants, cationic surfactants, structurants such as glucose, maleic acid and lactic acid, hair-conditioning compounds such as phospholipids, for example lecithin and cephalins; aromatic oils, dimethyl isosorbide and cyclodextrins; fiber-structure-improving active ingredients, in particular mono, di and oligosaccharides such as for example glucoses, galactoses, fructoses, fructoses and lactoses; dyes for coloring the agent; anti-dandruff active ingredients such as piroctone olamine, zinc omadine and climbazole; amino acids and oligopeptides; protein hydrolyzates of animal and/or plant basis, as well as in the form of its fatty-acid condensation products or optional anionically or cationically modified derivatives; plant oils; light stabilizers and UV blockers; active ingredients such as panthenol, pantothenic acid, pantolactone, allantoin, pyrrolidinone carboxylic acids and the salts thereof as well as bisabolol; polyphenols, in particular hydroxycinnamic acids, 6,7-dihydroxycoumarins, hydroxybenzoic acids, catechins, tannins, leucoanthocyanidins, anthocyanidins, flavanons, flavones and flavonols; ceramides or pseudoceramides; vitamins, provitamins and vitamin precursors; plant extracts; fats and waxes such as fatty alcohols, beeswax, montan wax and paraffins; swelling agents and penetration materials such as glycerol, propylene glycol monoethyl ether, carbonates, hydrogencarbonates, guanidines, ureas as well as primary, secondary and tertiary phosphates; opacifying agents such as latex, styrene/PVP and styrene/acrylamide copolymers; pearlescing agents such as ethylene glycol mono- and distearates as well as PEG-3-distearate; pigments as well as propellants such as propane-butane mixtures, N₂O, dimethylether, CO₂ and air.

The additional active ingredients and auxiliaries are used in the agents according to the invention preferably in quantities of from, in each case, 0.0001 to 10 wt.-%, in particular of from 0.0005 to 5 wt.-%, relative to the total weight of the application mixture.

As already mentioned, the application-ready oxidative color-changing agents will preferably be produced directly before the application from two or more separately packed preparations. This lends itself to separating ingredients, incompatible in particular for separation, in order to prevent a premature reaction. This lends itself in particular to a separation into multi-component systems, where the concerns relate to expectation or fear of incompatibilities of ingredients. In such systems, the application-ready agent is produced by the consumer by mixing the components directly before application. A coloring and/or brightening agent in which the oxidation dye precursors are present firstly separated from the oxidizing agent preparation, including preferably hydrogen peroxide, is preferred.

A preferred administration form of the agent according to the invention is a packaging unit (“kit of parts”) which, in separately manufactured containers, includes
at least one preparation (A) including in a cosmetic support at least one oxidation dye precursor according to formula (I) in a container A, and
at least one oxidizing agent preparation (B), including in a cosmetic support at least one oxidizing agent in a container B.

Preferably, the multi-component packaging unit (“kit of parts”) additionally includes instructions for use. Additionally, it may be preferred if furthermore an application aid, such as for example a comb or a brush, and/or personal protective equipment, such as for example disposable gloves, is added to the kit.

What has been said in respect of the agents according to the invention applies, mutatis mutandis, in respect of further preferred embodiments of the multi-component packaging unit (“kit of parts”).

The actual hair coloring agent is expediently produced directly before application by mixing preparations (A) and (B) as well as optionally (C). The application temperatures may lie in a range between 15 and 40° C. The hair coloring agent is removed by washing the hair to be colored after an exposure time of 5 to 45 minutes. Rewashing with a shampoo is dispensed with if a strongly surfactant-containing support, e.g. a color shampoo, has been used.

A further subject matter of the present invention is therefore a method for oxidative changing of the color of human hair, in which an agent according to the invention is applied to the hair according to the above specification, is left on the hair for an exposure time of from 5 to 45 minutes, preferably of from 8 to 35 minutes, and is rinsed out, or washed out of the hair with a shampoo.

While the fibers are being exposed to the agent, it can be advantageous to support the dyeing process by applying heat. Heat can be applied by an external heat source, such as e.g. hot air from a hot-air blower, and also, in particular in the case of dyeing the hair of a living subject, by the body temperature of the subject. In the latter possibility, conventionally the part to be dyed is covered with a cap. In particular the temperature during the exposure time is between 10° C. and 45° C., in particular between 20° C. and 40° C. At physiologically compatible temperatures of below 45° C., the coloring agents according to the invention already produce intensive dyes. Therefore they are suitable particularly for coloring human hair.

What has been said in respect of the agents according to the invention applies, mutatis mutandis, in respect of further preferred embodiments of the method according to the invention.

In summary, the present invention is outlined in particular by the following points:

1. An agent for oxidative changing of the color of keratin fibers, in particular human hair, including, in a cosmetic support, as a developer-type oxidation dye precursor, at least one compound of formula (I)

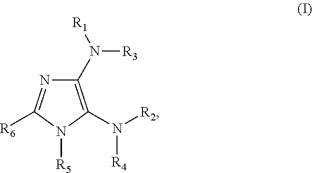

in which
R₁, R₂ independently of one another stand for hydrogen, a C₁-C₆ alkyl group, a C₂-C₆ alkenyl group, a hydroxy C₁-C₆ alkyl group, a polyhydroxy C₂-C₆ alkyl group, a C₁-C₆-alkyloxy C₂-C₆ alkyl group, an amino C₁-C₆ alkyl group, a polyamino C₂-C₆ alkyl group, a di(C₁-C₆-alkyl)amino C₁-C₆ alkyl group, a C₁-C₆ alkanoyl group, an aryl group or an aryl C₁-C₆ alkyl group,
R₃, R₄ independently of one another stand for hydrogen, a C₁-C₆ alkyl group, a C₂-C₆ alkenyl group, a hydroxy C₁-C₆ alkyl group, a polyhydroxy C₂-C₆ alkyl group, a C₁-C₆-alkyloxy C₂-C₆ alkyl group, an amino C₁-C₆ alkyl group, a polyamino C₂-C₆ alkyl group, a di(C₁-C₆-alkyl)amino C₁-C₆ alkyl group, a C₁-C₆ alkanoyl group, an aryl group or an aryl C₁-C₆ alkyl group,
R₅, R₆ independently of one another stand for hydrogen, a C₁-C₆ alkyl group, a C₂-C₆ alkenyl group, a hydroxy C₁-C₆ alkyl group, an aryl group or an aryl C₁-C₆ alkyl group,
and/or the physiologically compatible salt thereof.

2. The agent according to point 1, characterized in that it includes at least one compound of formula (I), in which R1 and R2 independently of one another stand for hydrogen or a C₁-C₆ alkanoyl group.

3. The agent according to any one of points 1 to 2, characterized in that it includes at least one compound of formula (I) in which R1 and R2 both stand for hydrogen.

4. The agent according to any one of points 1 to 3, characterized in that it includes at least one compound of formula (I), in which R₃ and R₄ independently of one another stand for hydrogen, a C₁-C₆ alkyl group or a C₁-C₆ alkanoyl group.

5. The agent according to any one of points 1 to 4, characterized in that it includes at least one compound of formula (I) in which R5 stands for hydrogen or for a C₁-C₆ alkyl group.

6. The agent according to any one of points 1 to 5, characterized in that it includes at least one compound of formula (I) in which R5 stands for a methyl group or an ethyl group.

7. The agent according to any one of points 1 to 6, characterized in that it includes at least one compound of formula (I) in which R6 stands for hydrogen.

8. The agent according to any one of points 1 to 7, characterized in that it includes at least one compound of formula (I), which is selected from the group of
1H-imidazole-4,5-diamine

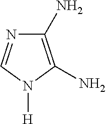

1-methyl-1H-imidazole-4,5-diamine

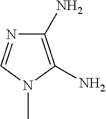

1-ethyl-1H-imidazole-4,5-diamine

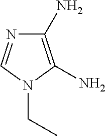

4-N-methyl-1H-imidazole-4,5-diamine

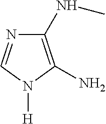

1,4-N-dimethyl-1H-imidazole-4,5-diamine

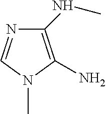

1-ethyl-4-N-methyl-1H-imidazole-4,5-diamine

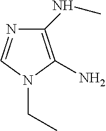

4-N-ethyl-1H-imidazole-4,5-diamine

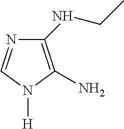

4-N-ethyl-1-methyl-1H-imidazole-4,5-diamine

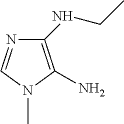

1,4-N-diethyl-1H-imidazole-4,5-diamine

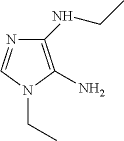

5-N-methyl-1H-imidazole-4,5-diamine

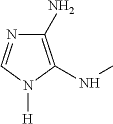

1,5-N-dimethyl-1H-imidazole-4,5-diamine

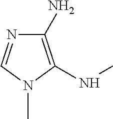

1-ethyl-5-N-methyl-1H-imidazole-4,5-diamine

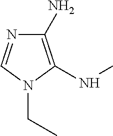

5-N-ethyl-1-H-imidazole-4,5-diamine

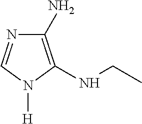

5-N-ethyl-1-methyl-1H-imidazole-4,5-diamine

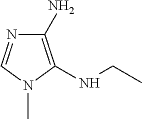

1,5-N-diethyl-1H-imidazole-4,5-diamine

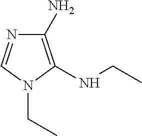

4-N,5-N-dimethyl-1H-imidazole-4,5-diamine

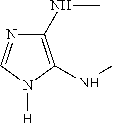

1,4-N,5-N-triemethyl-1H-imidazole-4,5-diamine

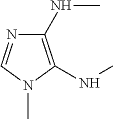

1-ethyl-4-N,5-N-dimethyl-1H-imidazole-4,5-diamine

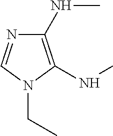

4-N-ethyl-5-N-methyl-1H-imidazole-4,5-diamine

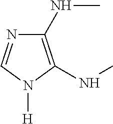

N-4-ethyl-1,5-N-dimethyl-1H-imidazole-4,5-diamine

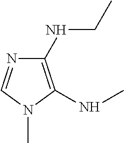

1,4-N-diethyl-5-N-methyl-1H-imidazole-4,5-diamine

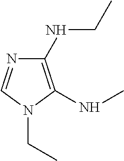

5-N-ethyl-4-N-methyl-1H-imidazole-4,5-diamine

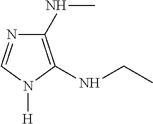

5-N-ethyl-1,4-N-dimethyl-1H-imidazole-4,5-diamine

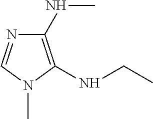

1-5-N-diethyl-4N-methyl-1H-imidazole-4,5-diamine

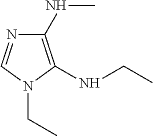

4-N,5-N-diethyl-1H-imidazole-4,5-diamine

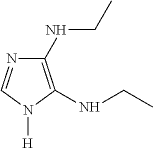

4-N,5-N-diethyl-1-methyl-1H-imidazole-4,5-diamine

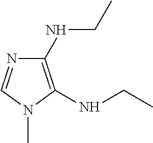

1,4-N,5-N-triethyl-1H-imidazole-4,5-diamine

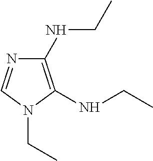

N-(5-amino-1H-imidazole-4-yl)acetamide

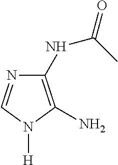

N-(5-amino-1-methyl-1H-imidazole-4-yl)acetamide

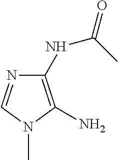

N-(5-amino-1-ethyl-1H-imidazole-4-yl)acetamide

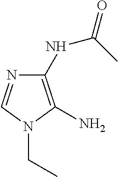

N-(4-amino-1H-imidazole-5-yl)acetamide

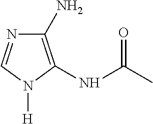

N-(4-amino-1-methyl-1H-imidazole-5-yl)acetamide

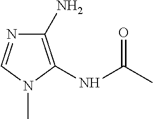

N-(4-amino-1-ethyl-1H-imidazole-5-yl)acetamide

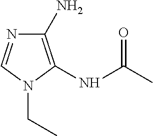

N-acetyl-N-(5-amino-1H-imidazole-4-yl)acetamide

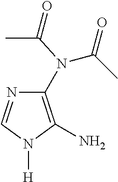

N-acetyl-N-(5-amino-1-methyl-1H-imidazole-4-yl)acetamide

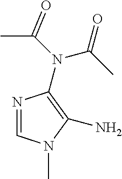

N-acetyl-N-(5-amino-1-ethyl-1H-imidazole-4-yl)acetamide

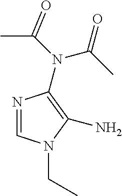

N-acetyl-N-(4-amino-1H-imidazole-5-yl)acetamide

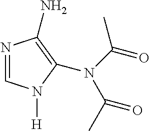

N-acetyl-N-(4-amino-1-methyl-1H-imidazole-5-yl)acetamide

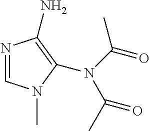

N-acetyl-N-(4-amino-1-ethyl-1H-imidazole-5-yl)acetamide

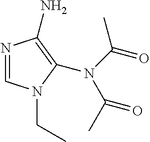

and/or the physiologically compatible salts of these compounds.

9. The agent according to any one of points 1 to 8, characterized in that it includes one or more compounds of formula (I) in a total quantity of from 0.001 to 5.0 wt.-%, preferably of from 0.025 to 2.5 wt.-%, particularly preferably of from 0.05 to 2.0 wt.-% and in particular preferably of from 0.1 to 1.5 wt.-%, relative to the total weight of the agent.

10. The agent according to any one of points 1 to 9, characterized in that it additionally includes at least one coupler-type oxidation dye precursor, which is selected from the group of 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)-amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholine-4-yl phenyl)amino]ethanol, 3-amino-4-(2-methoxyethoxy)-5-methylphenylamine, 1-amino-3-bis-(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5 -diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindolin, 6-hydroxyindolin and/or 7-hydroxyindolin and the physiologically compatible salts thereof.

11. The agent according to any one of points 1 to 10, characterized in that it additionally includes at least one developer-type oxidation dye precursor which is selected from the group of p-phenylenediamine, p-toluenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3-(1H-imidazole-1-yl)propyl]amine, N,N′-bis-(2-hydroxyethyl)-N,N′-bis-(4-aminophenyl)-1,3-diamino-propane-2-ol, bis-(2-hydroxy-5-aminophenyl)methane, 1,3-bis-(2,5-diaminophenoxy)-propane-2-ol, N,N′-bis-(4-aminophenyl)-1,4-diazacycloheptane, 1,10-bis-(2,5-diaminophenyl)-1,4,7,10-tetraoxadecane, 4-aminophenol, 4-amino-3-methylphenol, 4-amino-2-aminomethylphenol, 4-amino-2-(1,2-dihydroxyethyl)phenol and 4-amino-2-(diethylaminomethyl)phenol, 4,5-diamino-1-(2-hydroxyethyl)pyrazol, 2,4,5,6-tetraaminopyrimidine, 4-hydroxy-2,5,6-triaminopyrimidine, 2-hydroxy-4,5,6-triaminopyrimidine, as well as the physiologically compatible salts of these compounds. Particularly preferred additional developer components are p-toluenediamine, 2-(2-hydroxyethyl)-p-phenylenediamine, 2-methoxymethyl-p-phenylenediamine, N-(4-amino-3-methylphenyl)-N-[3-(1H-imidazole-1-yl)propyl]amine, 4,5-diamino-1-(2-hydroxyethyl)-pyrazol and the physiologically compatible salts thereof.

12. The agent according to any one of points 1 to 11, characterized in that it additionally includes hydrogen peroxide.

13. The agent according to any one of points 1 to 12, characterized in that it includes at least one compound which is selected from the group of ethylenediaminetetraacetic acid (EDTA), N-hydroxyethyl ethylenediaminetriacetic acid, diethylenetriaminepentaacetic acid (DTPA), ethylenediaminedisuccinic acid (EDDS), N,N-di-(2-hydroxyethyl)glycine, ethylenediaminediglutaric acid (EDGA), 2-hydroxypropylendiaminedisuccinic acid (HPDS), glycinamide-N,N′-disuccinic acid (GADS), ethylenediamine-N-N′-diglutaric acid (EDDG), 2-hydroxypropylenediamine-N-N′-disuccinic acid (HPDDS), diaminoalkyldi-(sulfosuccinic acid) (DDS), ethylenedicysteic acid (EDC), ethylenediamine-N-N′-bis(ortho-hydroxyphenyl) acetic acid (EDDHA), dipicolinic acid, 1-hydroxyethane-1,1-diphosphonic acid (HEDP), ethylenediaminetetra(methylenephosphonic acid) (EDTMP), diethylene-triaminepenta(methylenephosphonic acid) (DTPMP) and/or the physiologically compatible salts thereof.

14. The agent according to any one of points 1 to 13, characterized in that it includes water and a pH in the range of from 7.5 to 12.5, preferably of from 8.0 to 12.0, further preferably of from 8.5 to 11.5 and quite particularly preferably of from 9.0 to 10.5.

15. The agent according to any one of points 1 to 14, characterized in that it includes at least one alkalization agent from the group of ammonia, 2-aminoethane-1-ol (monoethanolamine), 2-amino-2-methylpropane-1-ol, 2-amino-2-methyl-propane-1,3-diol, triethanolamine, arginine, histidine and/or lysine.

### EXAMPLES

### Synthesis Example 1

### Synthesis of 1-methyl-1H-imidazole-4,5-diamine, dihydrochloride (E1)

1.1.1. Synthesis of 1-methyl-5-chlorimidazole

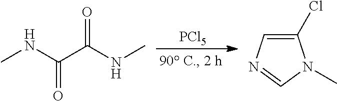

Phosphorus pentachloride (383.2 g, 1.8 mol) was added to methyl oxamide (116.1 g, 1.0 mol) and heated. A melt was created at 60° C. accompanied by the formation of foam. The mixture was stirred for 2 h between 85° C.-95° C., then the resulting POCl₃ was distilled. The dark-brown residue was dissolved in 700 ml H₂O and set to pH 8 with 915 g 12.75% KOH solution. The solution was extracted 4 times with 500 ml DCM, the combined organic extracts were dried over sodium sulfate, reduced and the obtained red-brown liquid subjected to a vacuum distillation. 1-methyl-5-chlorimidazole was obtained as a colorless liquid (53.1 g, 46%).
Flash point: 85-86° C. at 22 mbar
¹H NMR (400 MHz, d6-DMSO): δ=7.33 (s, 1H, 4-H), 6.80 (s, 1H, 2-H), 3.47 (s, 3H, CH₃)
¹³C NMR (100 MHz, d6-DMSO): δ=137.4 (C-2), 126.0 (C-4), 118.4 (C-5), 31.6 (CH₃).
   1.1.2. Synthesis of 5-chloro-1-methyl-4-nitro-imidazole

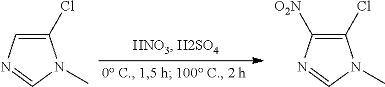

A solution of 1-methyl-5-chlorimidazole (57.0 g, 0.50 mol) and diluted nitric acid (690 ml, 1.15 mol) was concentrated to dryness. The obtained light-yellow oil (131.40 g) was added dropwise over 85 min in a nitrogen atmosphere to 214 ml ice-cold, concentrated sulfuric acid. Stirring subsequently took place for 2 h at 100° C. in a nitrogen atmosphere. The reaction solution was poured onto 21 of ice and the resultant precipitate was sucked out. This was washed neutral with ice water. 1-methyl-5-chloro-4-nitro-imidazole was obtained as white crystals (45.3 g, 62%).
Melting point: 147.5-148.9° C.
¹H NMR (400 MHz, d6-DMSO): δ=7.50 (s, 1H, 2-H), 3.72 (s, 3H, CH₃)
¹³C NMR (100 MHz, d6-DMSO): δ=142.4 (C-4), 134.6 (C-2), 119.6 (C-5), 32.9 (CH₃).
   1.1.3. Synthesis of 3-methyl-5-nitro-imidazole-4-amine

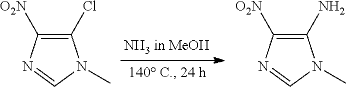

A suspension of 1-methyl-5-chloro-4-nitro-imidazole (40.4 g, 0.25 mol), methanol (250 ml) and ammonia in methanol (7 N, 214 ml, 1.50 mol) was heated to 130 ° C. in the autoclave for 24 h. In so doing, the pressure rose to 9.2 bar. The mixture was left to stir overnight at room temperature. The black suspension was sucked off and the obtained residue was boiled out 4 times with water at 80° C., filtered and the filtrate concentrated to dryness. 3-methyl-5-nitro-imidazole-4-amine was obtained as red-brown crystals (6.60 g, 19%).
200-300° C. sublimation, 314.2-315.7° C. decomposition
¹H NMR (400 MHz, d6-DMSO): δ=7.33 (s, 1H, 4-H), 7.22 (s, 1H, 2-H), 3.46 (s, 3H, CH₃)
¹³C NMR (100 MHz, d6-DMSO): δ=144.2 (C-4), 132.7 (C-2), 128.6 (C-5), 31.0 (CH₃).
   1.1.4. Synthesis of 1-methyl-1H-imidazole-4,5-diamine, dihydrochloride (E1)

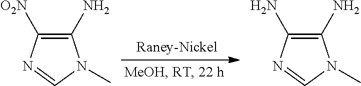

3-methyl-5-nitro-imidazole-4-amine (2.00 g, 14.1 mmol) was partly dissolved at 40° C. in 560 ml methanol and 240 ml water and filtered (RS: 0.9 g, 6.3 mmol). 5% palladium on carbon (0.47 g) was added to the solution and stirred for 23 h in the autoclave at 40° C. and 3 bar.

The catalyst was then filtered off, the solution poured over diluted hydrochloric acid (10%) and the solution reduced.

### 2. Dyeing Examples

### 2.1 Production of the Dye Cream

The following dye cream was produced:

| | | |
|---|---|---|
| Hydrenol D | 8.5 | wt.-% |
| Lorol tech. | 2.0 | wt.-% |
| Texapon NSO UP | 20.0 | wt.-% |
| Dehyton K | 12.5 | wt.-% |
| Eumulgin B2 | 0.75 | wt.-% |
| Sodium sulfite | 1.0 | wt.-% |
| Ammonium sulfate | 1.0 | wt.-% |
| Developer component E1 | 3 | mmol |
| Coupling component | 3 | mmol |
| Water | up to 100 | wt.-% |

Raw materials used:

| | |
|---|---|
| Hydrenol | C₁₂-C₁₈ fatty alcohol, (INCI name: cetearyl alcohol; |
| D | Cognis) |
| Lorol, | C₁₂-/C₁₈ fatty alcohol, (INCI name: cetearyl alcohol; |
| techn. | Cognis) |
| Texapon | lauryl alcohol diglycol ether sulfate, Na-salt (28% solution) |
| NSO UP | (INCI name: sodium laureth sulfate; Cognis) |
| Dehyton | Coco-betaine (INCI name: Cocamidopropyl betaine; |
| K | Cognis) |
| Eumulgin | C₁₆-/C₁₈ fatty alcohol, ethoxylated (20 EO) (INCI name: |
| B2 | Ceteareth-10; Cognis) |

Hydrenol D and Lorol, techn. were melted together with Texapon NSO-UP, Dehyton K and Eumulgin B2 at 80° C. Then, the melt was emulsified with the sodium sulfite dissolved in a part of the water and ammonium sulfite. The developer according to the invention was dissolved, accompanied by heating, in propylene glycol and a further part of the indicated quantity of water and added accompanied by stirring. The coupler was likewise dissolved in a part of the indicated quantity of water and added accompanied by stirring. The formulation was then filled to 100% with water and stirred cold.

The dye cream obtained in this way was mixed in a 1:1 ratio with the following developer dispersion with a hydrogen peroxide content of 6%. A pH of 10 was set by adding ammonia.

| | | |
|---|---|---|
| Dipicolinic acid | 0.1 | wt.-% |
| Sodium pyrophosphate | 0.03 | wt.-% |
| Turpinal SL | 1.50 | wt.-% |
| Texapon N28 | 2.00 | wt.-% |
| Acrysol 22 | 0.60 | wt.-% |
| Hydrogen peroxide, 50 wt.-% | 12.00 | wt.-% |
| Caustic soda solution, 45 wt.-% | 0.80 | wt.-% |
| Water | up to 100 | wt.-% |

Raw materials used:

| Turpinal | SL1-hydroxyethane-1,1-diphosphonic acid (approx. 58-61% |
|---|---|
| | active-ingredient content; INCI name: Etidronic Acid, Aqua |
| | (Water)) (Solutia) |
| Texapon | lauryl ether sulfate sodium salt (min. 26.5% active-ingredient |
| N28 | content; INCI name: Sodium Laureth Sulfate) (Cognis) |
| Acrysol | acryl polymer (approx. 29.5-30.5% solid in water; INCI name: |
| 22 | Acrylates/Steareth-20 methacrylate copolymer) |

A 4-fold quantity of the application-dye mixture was applied to a strand of 80% gray hair (Kerling) for the dyeing process. The strands were rinsed after an exposure time of 30 minutes at 32° C. and washed with a conventional shampoo. The coloration of the strands was visually evaluated after drying using a daylight lamp. The coloring results are summarized in the table below.

E1: Dyes with 1-methyl-1H-imidazole-4,5-diamine, dihydrochloride

| Example | Coupling component | Obtained hue/color intensity |
|---|---|---|
| E1-1 | Resorcinol | Dark brown/+++ |
| E1-2 | 2-methylresorcinol | Dark brown/++ |
| E1-3 | 4-chlororesorcinol | Dark brown/++ |

+++ high intensity
++ average intensity
+ low intensity

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An agent for oxidative changing of the color of keratin fibers, in particular human hair, including, in a cosmetic support, as a developer-type oxidation dye precursor, at least one compound of formula (I) 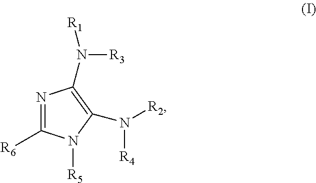
wherein
R₁, R₂ independently of one another stand for hydrogen, a C₁-C₆ alkyl group, a C₂-C₆ alkenyl group, a hydroxy C₁-C₆ alkyl group, a polyhydroxy C₂-C₆ alkyl group, a C₁-C₆-alkyloxy C₂-C₆ alkyl group, an amino C₁-C₆ alkyl group, a polyamino C₂-C₆ alkyl group, a di(C₁-C₆-alkyl)amino C₁-C₆ alkyl group, a C₁-C₆ alkanoyl group, an aryl group or an aryl C₁-C₆ alkyl group,
R₃, R₄ independently of one another stand for hydrogen, a C₁-C₆ alkyl group, a C₂-C₆ alkenyl group, a hydroxy C₁-C₆ alkyl group, a polyhydroxy C₂-C₆ alkyl group, a C₁-C₆-alkyloxy C₂-C₆ alkyl group, an amino C₁-C₆ alkyl group, a polyamino C₂-C₆ alkyl group, a di(C₁-C₆-alkyl)amino C₁-C₆ alkyl group, a C₁-C₆ alkanoyl group, an aryl group or an aryl C₁-C₆ alkyl group,
R₅, R₆ independently of one another stand for hydrogen, a C₁-C₆ alkyl group, a C₂-C₆ alkenyl group, a hydroxy C₁-C₆ alkyl group, an aryl group or an aryl C₁-C₆ alkyl group,
and/or a physiologically compatible salt thereof.

2. The agent according to claim 1, wherein the agent includes at least one compound of formula (I) in which R1 and R2 independently of one another stand for hydrogen or a C₁-C₆ alkanoyl group.

3. The agent according to claim 1, wherein the agent includes at least one compound of formula (I) in which R1 and R2 both stand for hydrogen.

4. The agent according to claim 1, wherein the agent includes at least one compound of formula (I) in which R₃ and R₄ independently of one another stand for hydrogen, a C₁-C₆ alkyl group or a C₁-C₆ alkanoyl group.

5. The agent according to claim 1, wherein the agent includes at least one compound of formula (I) in which R5 stands for hydrogen or for a C₁-C₆ alkyl group.

6. The agent according to claim 1, wherein the agent includes at least one compound of formula (I) in which R5 stands for a methyl group or an ethyl group.

7. The agent according to claim 1, wherein the agent includes at least one compound of formula (I) in which R6 stands for hydrogen.

8. The agent according to claim 1, wherein the agent includes at least one compound of formula (I), which is selected from the group consisting of
1H-imidazole-4,5-diamine 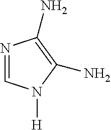
1-methyl-1H-imidazole-4,5-diamine 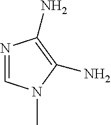
1-ethyl-1H-imidazole-4,5-diamine 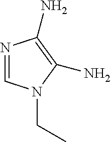
4-N-methyl-1H-imidazole-4,5-diamine 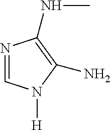
1,4-N-dimethyl-1H-imidazole-4,5-diamine 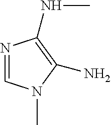
1-ethyl-4-N-methyl-1H-imidazole-4,5-diamine 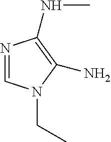
4-N-ethyl-1H-imidazole-4,5-diamine 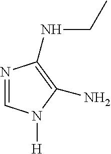
4-N-ethyl-1-methyl-1H-imidazole-4,5-diamine 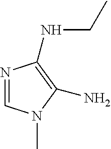
1,4-N-diethyl-1H-imidazole-4,5-diamine 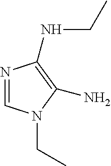
5-N-methyl-1H-imidazole-4,5-diamine 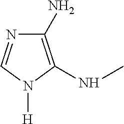
1,5-N-dimethyl-1H-imidazole-4,5-diamine 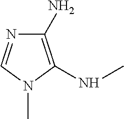
1-ethyl-5-N-methyl-1H-imidazole-4,5-diamine 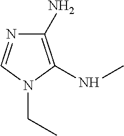
5-N-ethyl-1-H-imidazole-4,5-diamine 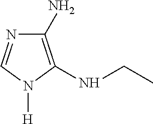
5-N-ethyl-1-methyl-1H-imidazole-4,5-diamine 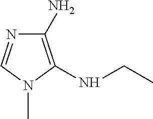
1,5-N-diethyl-1H-imidazole-4,5-diamine 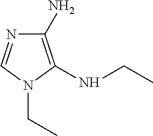
4-N,5-N-dimethyl-1H-imidazole-4,5-diamine 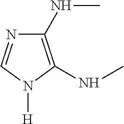
1,4-N,5-N-trimethyl-1H-imidazole-4,5-diamine 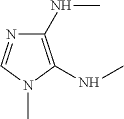
1-ethyl-4-N,5-N-dimethyl-1H-imidazole-4,5-diamine 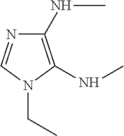
4-N-ethyl-5-N-methyl-1H-imidazole-4,5-diamine 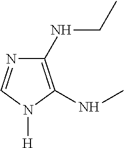
4-N-ethyl-1,5-N-dimethyl-1H-imidazole-4,5-diamine 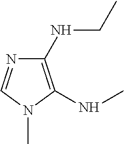
1,4-N-diethyl-5N-methyl-1H-imidazole-4,5-diamine 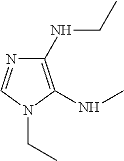
5-N-ethyl-4-N-methyl-1H-imidazole-4,5-diamine 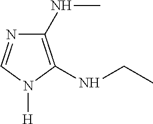
5-N-ethyl-1,4-N-dimethyl-1H-imidazole-4,5-diamine 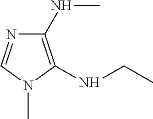
1-5-N-diethyl-4N-methyl-1H-imidazole-4,5-diamine 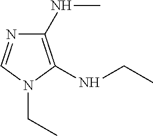
4-N, 5-N-diethyl-1H-imidazole-4,5-diamine 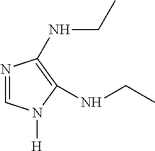
4-N, 5-N-diethyl-1-methyl-1H-imidazole-4,5-diamine 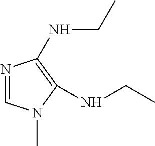
1,4-N, 5-N-triethyl-1H-imidazole-4,5-diamine 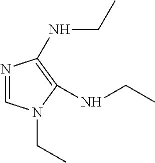
N-(5-amino-1H-imidazole-4-yl)acetamide 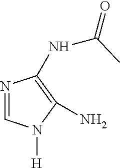
N-(5 -amino-1-methyl-1H-imidazole-4-yl)acetamide 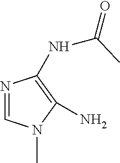
N-(5-amino-1-ethyl-1H-imidazole-4-yl)acetamide 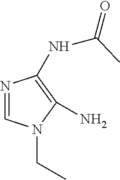
N-(4-amino-1H-imidazole-5-yl)acetamide 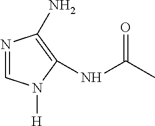
N-(4-amino-1-methyl-1H-imidazole-5-yl)acetamide 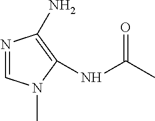
N-(4-amino-1-ethyl-1H-imidazole-5-yl)acetamide 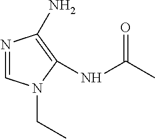
N-acetyl-N-(5-amino-1H-imidazole-4-yl)acetamide 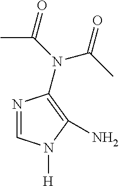
N-acetyl-N-(5-amino-1-methyl-1H-imidazole-4-yl)acetamide 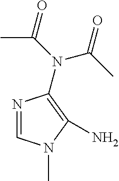
N-acetyl-N-(5-amino-1-ethyl-1H-imidazole-4-yl)acetamide 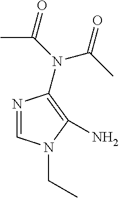
N-acetyl-N-(4-amino-1H-imidazole-5-yl)acetamide 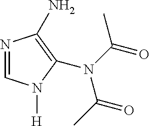
N-acetyl-N-(4-amino-1-methyl-1H-imidazole-5-yl)acetamide 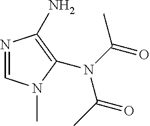
N-acetyl-N-(4-amino-1-ethyl-1H-imidazole-5-yl)acetamide 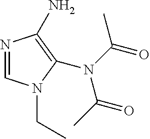
and the physiologically compatible salts of such compounds.

9. The agent according to claim 1, wherein the agent includes one or more compounds of formula (I) in a total quantity of from 0.001 to 5.0 wt.% relative to the total weight of the agent.

10. The agent according to claim 1, further including at least one coupler-type oxidation dye precursor selected from the group consisting of 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)-amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethyleneamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholine-4-yl phenyl)amino]ethanol, 3-amino-4-(2-methoxyethoxy)-5-methylphenylamine, 1-amino-3-bis-(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindolin, 6-hydroxyindolin, 7-hydroxyindolin, and physiologically compatible salts of such compounds.
